(19) United States
(12) Patent Application Publication
(10) Pub. No.: US 2013/0274224 A1
(43) Pub. Date: Oct. 17, 2013

(54) STERILE INJECTABLE AQUEOUS FORMULATION USED IN OPHTHALMOLOGY

(71) Applicant: Samuel Gavard Molliard, Bogeve (FR)

(72) Inventor: Samuel Gavard Molliard, Bogeve (FR)

(73) Assignee: ANTEIS S.A., Planles-Ouates (CH)

(21) Appl. No.: 13/993,281

(22) PCT Filed: Dec. 8, 2011
(86) PCT No.: PCT/IB11/55549

§ 371 Date: Jun. 11, 2013

(30) Foreign Application Priority Data
Dec. 17, 2010 (FR) FR10/60707

(51) Int. Cl. A61K 31/734; A61K 45/06; A61K 31/728

(52) U.S. Cl. CPC A61K 31/734; A61K 31/728; A61K 45/06
USPC 514/54

(57) ABSTRACT

The present invention relates to an intraocularly injectable sterile aqueous formulation based on a mixture of hyaluronic acid and alginate, or a salt thereof, used in ophthalmology and having specific viscoelasticity, spreading, covering and ocular tissue adhesion properties and also a high capacity for neutralizing free radicals, said properties enabling said composition to strongly protect the eye tissues.

### FIELD OF THE INVENTION

The present invention relates to an intraocularly injectable sterile aqueous formulation based on a mixture of hyaluronic acid and alginate, or a salt thereof, used in ophthalmology and having specific viscoelasticity, spreading, covering and ocular tissue adhesion properties and also a high capacity for neutralizing free radicals, said properties enabling said composition to strongly protect eye tissue.

### CONTEXT OF THE INVENTION

Any surgical invasion can damage eye tissue. In order to minimize the damage in question, especially in areas where the tissues are particularly fragile and/or irreplaceable, it is known to use viscoelastic formulations as surgical aids (see table 1). Such solutions protect the tissues from surgical instruments and assist in manipulating said tissues. They are also used for maintaining the spaces or volumes of the eye.

In the context of cataract surgery, considerable damage with respect to eye tissue and in particular of corneal endothelial cells can be observed. An excessive loss of these cells constitutes a serious complication of cataract surgery since this loss can result in an irreversible corneal edema (bullous keratopathy) which can then entail severe pain and permanent loss of vision.

Cataract surgery by phacoemulsification has become over the past decades the most popular technique. This technique entails a loss of corneal endothelial cells due to mechanical damage, heat emission and highly reactive free radicals generated by the ultrasound. In the literature, the harmful effects of free radicals is increasingly propounded as a major factor of endothelial loss (Murano N., Ishizaki, Corneal endothelial cell damage by free radicals associated with ultrasound oscillation, Arch. Ophthalmol., Vol. 126, 6, 816-820, 2008).

Currently, various viscoelastic formulations (see table 1) are available on the ophthalmology market. These formulations are marketed under various brand names and include options such as hyaluronic acid, hypromelose, hyaluronic acid with hypromelose or hyaluronic acid with chondroitin sulphate.

Each of these formulations has specific benefits to meet the needs of practitioners (protection of eye tissue, strong capacity for creating and maintaining intraocular spaces, strong capacity to be aspirated from the eye at the end of a surgical intervention, . . . ). Indeed, some formulations are known for their strong ability to protect eye tissue, notably corneal endothelial cells. This is the case of the product Viscoat® (marketed by Alcon Laboratories), which is a formulation on the basis of hyaluronic acid and chondroitin sulfate, or of other products on the basis of hypromelose such as OcuCoat® (marketed by Bausch+Lomb). Other products, such as Healon® or Healon® GV (both marketed by Abbott Medical Optics), are better known for their strong capacity for creating and maintaining intraocular spaces.

The interest of all these formulations for protecting eye tissue is well known. However, it has been shown in the literature that the capacity for protecting eye tissue and notably corneal endothelial cells is very different from one product to another (Takahashi H., Free radicals in phacoemulsification and aspiration procedures, Arch. Ophthalmol., Vol. 120, 1348-1352, 2009//Augustin A. J., Oxidative tissue damage after phacoemulsification: influence of ophthalmic viscosurgical devices, J. Cataract Refract Surg, Vol. 30, 424-427, 2004//Bresciani C., Lebuisson D. A., Eveillard M., Viscosité dynamique et protection endothéliale cornéenne du Healonid, du Healon GV, du Provisc et du Viscoat au cours de la phacoémulsification [Dynamic viscosity and corneal endothelial protection of Healonid, Healong GV, Provisc and Viscoat during phacoemulsification], J. Fr. Ophhtalmologie, 19, 1, 39-50, 1996).

In the literature, there are different theories to explain the ability of one viscoelastic formulation to achieve better protection of the eye tissue than another formulation. The most commonly articulated hypotheses are i) a greater dispersal ability of the product makes it possible to cover the surface of the eye tissue to be protected, ii) the eye tissue comprises CD44 hyaluronic acid receptors that ensure good adherence of a formulation based on hyaluronic acid to the surface of the eye tissue, iii) formulations with chondroitin sulfate, a polymer with a highly negative charge, exhibit strong adherence with eye tissue which is charged positively.

Thus, a formulation such as Viscoat®, known for its strong capacity to protect the corneal endothelium, is composed of hyaluronic acid, chondroitin sulfate and has a great dispersal ability. Thanks to its strong adherence with the eye tissue, this formulation is much more difficult to remove from the eye at the end of surgery as compared with a formulation containing only hyaluronic acid, as is the case of Healon®.

To date, there is no viscoelastic formulation for use in ophthalmology that has ideal characteristics enabling it to answer all the needs of the practitioners throughout surgery such as cataract surgery. Thus, many practitioners combine different products during their eye surgery to combine different viscoelasticity and eye protection characteristics in order to achieve the desired efficiency.

In this context, it is important to provide practitioners with formulations having characteristics adapted to their needs. It is notably essential to make available to them formulations having an optimum capacity for protecting eye tissue such as corneal endothelial cells, in order to limit complications associated with eye surgery.

### SUMMARY OF THE INVENTION

The object of the invention described hereafter is to propose a new intraocularly injectable sterile aqueous formulation based on a mixture of hyaluronic acid and alginate, or a salt thereof, used in ophthalmology and having specific viscoelasticity, spreading, covering and ocular tissue adhesion properties and also a high capacity for neutralizing free radicals, said properties enabling said composition to strongly protect eye tissue and notably corneal endothelial cells. This formation is characterized in that i) the concentration of hyaluronic acid, or a salt thereof, is comprised between 0.8% and 5% (weight/volume), and the molecular weight of the hyaluronic acid, or a salt thereof, is comprised between 4×10⁵ Da and 7×10⁶ Da, ii) the concentration of alginate, or a salt thereof, is comprised between 0.01% and 10% (weight/volume), and iii) the zero shear viscosity η₀ of said injectable sterile aqueous formulation is comprised between 5 Pa·s and 450 Pa·s.

### DETAILED DESCRIPTION OF THE INVENTION

The present invention concerns an intraocularly injectable sterile aqueous formulation used in ophthalmology based on a mixture of hyaluronic acid and alginate, or a salt thereof, characterized in that:
the concentration of hyaluronic acid, or a salt thereof, is comprised between 0.8% and 5% (weight/volume), and the molecular weight of the hyaluronic acid, or a salt thereof, is comprised between 4×10⁵ Da and 7×10⁶ Da,
the concentration of alginate, or a salt thereof, is comprised between 0.01% and 10% (weight/volume), and
the zero shear viscosity η₀ of said injectable sterile aqueous formulation is comprised between 5 Pa·s and 450 Pa·s.

The present invention, due to its composition and specific rheological properties, has a strong capacity for protecting eye tissue.

In a completely surprising manner, this formulation has remarkable viscoelasticity properties (a synergy in terms of the viscosity between the hyaluronic acid and the alginate but also in terms of the elastic module G′ and of the viscous module G″ can be observed). These properties notably enable an appropriate and homogenous spreading of the formulation on the eye tissue as well as the creation and maintaining of an intraocular space (which thus makes it possible to limit the “mechanical” aggressions that occur in the eye during surgery by creating a space for the surgeon to perform his manipulations). Furthermore, the combination between the hyaluronic acid and the alginate, in the conditions according to the invention, enables a good adherence between the formulation and the eye tissue, thus allowing a resistant “protective deposit” to be created at the surface of the eye (so that the “mechanical and free-radical aggressions” that occur in the eye during surgery can be limited).

It has also been observed that the presence of alginate in the formulation according to the invention makes it possible to reinforce the gel's ability to neutralize free radicals present in the ocular space and notably highly harmful free radicals released during cataract surgery by phacoemulsification. It must be noted that this strong anti-radical activity, which is key for limiting tissue aggressions by free radicals, can be improved by adding a molecule with antioxidant properties to the formulation according to the invention, such as a molecule from the polyol family.

It has been observed that the intraocularly injectable sterile aqueous formulation of the invention exhibits a synergy in terms of viscosity between the hyaluronic acid and the alginate (synergy in terms of the viscosity but also in terms of the elastic module G′ and of the viscous module G″). Indeed, the presence of alginate (or a salt thereof) in a solution based on hyaluronic acid (or a salt thereof) causes a significant increase of the viscosity relative to a solution of hyaluronic acid alone. This increase cannot be explained by the viscosity of the alginate solution alone, as shown in the examples 1, 2 and 3 of the present description. In fact, the viscosity of a viscoelastic aqueous formulation (or hydrogel) comprising hyaluronic acid and alginate, in the conditions of the invention, is significantly greater than the sum of the viscosities corresponding to a 1^{st} formulation comprising hyaluronic acid and to a 2^{nd} formulation comprising alginate (it must be noted that a formulation based on alginate alone, i.e. without hyaluronic acid, exhibits insufficient viscosity for use in ophthalmology for efficiently maintaining the intraocular space).

The same synergy can be observed in terms of the elastic modulus G′ and of the viscous modulus G″. This property is observed for formulations according to the invention comprising or not comprising antioxidant(s) such as a polyol.

This synergy, demonstrating a highly specific structure between the hyaluronic acid and the alginate, is of major importance in the field of ophthalmology since viscoelasticity plays a key role in the product's efficiency, notably among others on the ability to create and maintain the intraocular space and on the ability to spread over the eye tissue. It is for this reason that intraocular viscoelastic products (products having a viscous component and an elastic component, used by injection into the eye) used in the frame of cataract surgery are classified according to their level of viscosity (see Dick., et al., in Ophthalmologe 1999 March; 96(3):193-211 and the following Table 1).

TABLE 1

| Product | Zero shear viscosity η₀ |
|---|---|
| Amivisc Plus ® | 128 |
| AMO Vitrax ® | 41 |
| Biolon ® | 243 |
| Dispasan ® | 130 |
| Dispasan Plus ® | 782 |
| Healon ® | 243 |
| Healon GV ® | 2451 |
| Healon 5 ® | 5525 |
| Microvisc (Morcher Oil) ® | 1162 |
| Microvisc Plus ® | 3663 |
| Morcher Oil ® | 1253 |
| Provisc ® | 207 |
| Rayvisc ® | 78 |
| Viscoat ® | 58 |
| Viscorneal (Allervisc) ® | 733 |
| Viscorneal Plus (Allervisc Plus) ® | 1176 |
| Visko ® | 206 |
| Visko Plus ® | 1683 |
| Acrivisc ® | 7 |
| Adatocel ® | 8 |
| Coatel ® | 6 |
| HPMC Ophthal H ® | 94 |
| HPMC Ophthal L ® | 7 |
| Ocucoat ® | 6 |
| PeHa-Visko ® | 5 |
| Visco Shield ® | 60 |

The “zero shear viscosity” refers to the viscosity of the hydrogel when the latter is at rest (no shear stress).

The elastic modulus that represents the elastic behavior of the material for a given frequency is classically denoted as G′, whilst the viscous modulus, which represents the viscous behavior of the material for a given frequency, is conventionally denoted G″. These values have notably been defined in the “Handbook of Pressure Sensitive Adhesive Technology” 3rd edition, D. Satas, chap. 9 p. 155 to 157.

The formulation according to the invention has significant spreading, covering and ocular tissue adhesion properties in the presence of a low concentration of alginate. Indeed, the concentration of alginate, or of a salt thereof, is advantageously comprised between 0.01% and 10% (weight/volume). In a preferred manner, this concentration is comprised between 0.01% and 5% (weight/volume).

The presence of alginate in the formulation according to the invention makes it possible to achieve remarkable surface properties for ophthalmology (strong adhesion to the eye tissue), even at a very low concentration. According to one embodiment of the invention, the alginate and the hyaluronic acid (or a salt thereof) are structurally associated or combined as described in the literature (Oerther et al., Biochim Biophys Acta. 1999 Jan. 4; 1426(1):185-94; Oerther et al., Biopolymers. 2000 Oct. 5; 54(4):273-81).

Among the alginate salts preferred according to the invention, alginate salts with a cation can be mentioned, for example a monovalent or divalent salt such as sodium, potassium, magnesium, calcium or manganese salt. Sodium salts are particularly preferred.

For a formulation according to the invention, a strong adhesion between said formulation and the eye tissue has been observed. This strong adhesion is key for the protection of the eye during surgery and notably for limiting the loss of endothelial cells during cataract surgery. This strong adherence, coupled with the inventive hydrogel's capacity for spreading and covering the eye tissue, allows the formation of a “protective deposit” at the surface of the tissue. This “protective deposit” enables the eye cells to be protected by limiting the “mechanical and free-radical aggressions” suffered by the eye tissue during surgery.

The strong adhesion observed with the combination [hyaluronic acid/alginate] according to the invention is significantly greater than that obtained with a formulation on the basis of hyaluronic acid without alginate (see example 5).

This strong adherence between the hyaluronic acid and the alginate is also observed when the formulation is to be removed from the intra-ocular space by aspiration. The aspiration time, just as the Viscoat® product, is greater than that of Healon® (formulation based on hyaluronic acid).

Without being bound to a theoretical explanation, the multiple negative charges (twice as many negative charges than hyaluronic acid) carried by the alginate in the formulation according to the invention must strongly contribute to this good formulation-to-tissue adherence, as the eye tissue are charged positively.

It has been observed that this good formulation-to-tissue adherence is also correlated with the viscosity of the formulation according to the invention (see example 5). Thus, the zero shear viscosity η₀ of said intraocularly injectable sterile aqueous formulation according to the invention is generally comprised between 5 Pa·s and 450 Pa·s, in order to ensure adequate wetting for the formulation's compounds to interact with the eye tissue. In order to achieve excellent formulation-to-tissue adherence, it is preferably for the zero shear viscosity η₀ to be comprised between 5 to 90 Pa·s. A good formulation-to-tissue adherence is observed for a viscosity of the formulation according to the invention preferably comprised between 90 Pa·s and 160 Pa·s. Furthermore, a satisfactory adherence is observed for a viscosity of the inventive formulation comprised between 160 Pa·s and 450 Pa·s.

It has also been observed that the formulation according to the invention has a strong ability to neutralize the free radicals generated in the eye whilst significantly limiting the loss of its rheological properties due to degradation caused by free radicals. As described in the literature, free radicals have a highly harmful effect on the cells of the eye tissue. Free radicals are generated notably during the phacoemulsification phase during cataract surgery.

The inventive formulation, thanks to its great ability to neutralize free radicals, enables the quantity of free radicals present in the eye to be reduced and thus the damage caused by these radicals on the eye tissue to be limited. As demonstrated in example 6, the presence of alginate in the formulation according to the invention makes it possible to achieve a resistance to degradation due to free radicals superior to that of a formulation based on hyaluronic acid.

This improved ability to neutralize free radicals and maintain its rheological properties is a key characteristic of this new formulation to provide strong protection of the eye tissue. According to a particular embodiment of the invention, this characteristic can be improved by adding one or several antioxidants to the inventive formulation. This antioxidant is advantageously a polyol.

In addition to enhancing the formulation's antioxidant capacity, polyol, thanks to its ability to form weak bonds with hyaluronic acid and alginate, is also capable of interacting by weak bonds with the eye tissue. The creation of weak bonds between the formulation and the eye tissue through the polyol of the inventive formulation can contribute to increasing the strong adherence between formulation and eye tissue.

Hyaluronic acid is a glycosaminoglycan distributed widely among connective, epithelial and neural tissues. It constitutes one of the main components of the extracellular matrix. It contributes significantly to the proliferation and migration of cells. Hyaluronic acid is a polymer of disaccharides which themselves are composed of D-glucoronic and N-N-acetylglucosamine acid, connected to one another by alternating glycosidic beta-1,4 and beta-1,3 bonds. The molecular weight of the hyaluronic acid in vivo can amount to up to approx. 2 to 7 million daltons.

The present invention generally comprises a concentration of hyaluronic acid, or a salt thereof, between 0.8% and 5% (weight/volume), preferably between 1% and 4%.

Among the hyaluronic acid salts preferred according to the invention, salts with a cation can be mentioned, for example a monovalent or divalent salt such as sodium, potassium, magnesium, calcium or manganese salt. Sodium salts are particularly preferred.

Advantageously, the aqueous formulation according to the invention comprises hyaluronic acid, or a salt thereof, whose molecular weight is comprised between 4×10⁵ Da and 2×10⁶ Da. According to a preferred embodiment, the molecular weight of the hyaluronic acid, or a salt thereof, is comprised between 4×10⁵ Da and 1.5×10⁶ Da.

Also according to a preferred embodiment, the molecular weight of the alginate, or a salt thereof, is less than 3×10⁵ Da. According to a particularly preferred variant, the molecular weight of the alginate, or a salt thereof, is less than 1×10⁵ Da.

An alternative variant of the present invention provides that the range of molecular weight of the hyaluronic acid, or a salt thereof, comprised between 4×10⁵ Da and 2×10⁶ Da, is obtained by a mixture of different molecular weights of hyaluronic acid or of alginate (or a salt thereof). In this case, the range of molecular weight consider thus reflects the result of the average of molecular weights of two or several hyaluronic acids and/or alginates or a salt thereof.

The hyaluronic acid, or a salt thereof, as well as the alginate, or a salt thereof, can be in the form of linear and/or reticulated and/or graft polymer.

Reticulation processes, notably of hyaluronic acid, are known to the one skilled in the art and have been described for example in applications WO 2005/085329 (fled in the name of ANTEIS SA) and WO 97/004012 (filed in the name of Q MED AB).

In the frame of advantageous embodiments, the aqueous formulation according to the invention further comprises one or several antioxidants. In a preferred manner, these antioxidants are chosen from the polyol family. Among the most commonly encountered polyols, it is possible to mention among others sorbitol, glycerol, mannitol and glycol propylene. According to a particularly advantageous variant, the polyol concentration is less than 10% (weight/volume), preferably less than 5% (weight/volume).

Also in the frame of advantageous embodiments, the aqueous formulation according to the invention further comprises an anesthetic. Among the most commonly encountered anesthetics, the following among other examples can be mentioned: lidocaine alone or in combination with adrenalin, procaine, etidocaine alone or in combination with adrenaline, articaine alone or in combination with adrenaline, mepivacaine, pramocaine, quinisocaine or one or several of these anesthetics.

One object of the present invention relates to the use of the aqueous formulations described here above as aids and/or temporary implants for surgery in ophthalmology.

The aqueous formulations of the invention are particularly effective in this context of use, in particular during cataract surgery. In such a context of use, the aqueous formulations according to the invention are injected, fulfill their function as described above and in the examples and are then removed totally or largely after surgery.

The aqueous formulations of the invention can also not be removed after surgery. They can thus play a key role in improving the clinical outcome of surgery as in the case of glaucoma surgery, for which implantation in the eye of a formulation according to the invention can enable a drainage between different compartments of the eye to be provided and thus improve postoperative fibrosis, thereby achieving a better success rate of the surgery.

Another object of the present invention also concerns a formulation for hydrating and/or healing and/or protecting eye tissue as used in ophthalmology, characterized in that it consists, or consists essentially, of an aqueous formulation according to the invention described above. The formulation according to the invention can then be used either in the context of eye surgery or outside of eye surgery.

The present invention also comprises an aqueous formulation, such as described here above, for the treatment of ophthalmic diseases such as retinal degeneration, glaucoma, cataract, for intraocular use.

The aqueous formulations of the invention are generally used as such but it is not excluded that at least one other additive (other than those mentioned here above) and/or at least one active ingredient is added thereto. Consequently, the implants and/or aids mentioned here above thus consist, or consist essentially, of the formulations according to the invention.

According to another object, the present invention concerns a method for preparing an aqueous formulation according to the invention, having the following steps:
a) In a first step, adequate quantities of hyaluronic acid and alginate polymers or a salt thereof are put into a solution (solubilization of the polymers simultaneously or successively), so that the concentration of hyaluronic acid, or a salt thereof, is comprised between 0.8% and 5% (weight/volume), preferably between 1% and 4%, and the concentration of alginate, or a salt thereof, is comprised between 0.01% and 10% (weight/volume), preferably between 0.01% and 5%.
b) In a second step, a suitable mixture is made.
c) In a third step, the mixture thus obtained is filled in its final container such as for example inside a syringe.
d) In a fourth step, the product is sterilized.

Filling the formulation into its container as detailed in step c) can also be carried out after the sterilization step. In this case, the filling of the product should be done aseptically.

Sterilizing the formulation according to step d) of the invention is carried out according to the various techniques known by the one skilled in the art. Examples include aseptic filtration or moist heat sterilization, the latter being preferred. The one skilled in the art will be able to select a sterilization cycle at the temperature (temperature and duration of the sterilization cycle) appropriate for the sterilization of the product. For example, the following moist heat sterilization cycles can be used: 131° C., 1 min/130° C., 3 min/125° C., 7 min/121 ° C., 20 min/121° C., 10 min/100° C., 2 h.

According to another object, the present invention relates to a kit preferably in the form of a syringe containing the formulation such as described above.

The present invention also relates to a kit in the form of a container different from a syringe, such as an ampoule or vial containing the formulation as described above.

The invention will now be illustrated by way of non-limiting example with the following examples 1 to 6.

### EXAMPLES

### Example 1

### Highlighting the HA/Alginate Synergy in the Formulation According to the Invention

Preparation of three hydrogels according to the method described below:

### Hydrogel A:

In 30 ml iso-osmolar aqueous solution, 0.90 g sodium hyaluronate at 1.1 MDa is added.

The preparation is mixed by mechanical agitation at room temperature for 24 hours. A visually transparent and homogenous gel is obtained. Let A1 be the resulting gel.

The gel thus obtained is filled into a glass syringe and sterilized by autoclave during 20 min at a temperature of 121° C. Let A2 be the resulting gel.

### Hydrogel B:

In 30 ml iso-osmolar aqueous solution, 0.6 g sodium alginate at 35 000 Da is added.

The preparation is mixed by mechanical agitation at room temperature for 24 hours. A visually transparent and homogenous gel is obtained. Let B1 be the resulting gel.

The gel thus obtained is filled into a glass syringe and sterilized by autoclave during 20 min at a temperature of 121° C. Let B2 be the resulting gel.

### Hydrogel C:

In 30 ml iso-osmolar aqueous solution, 0.90 g sodium hyaluronate at 1.1 MDa and 0.6 g sodium alginate at 35 000 Da are added.

The preparation is mixed by mechanical agitation at room temperature for 24 hours. A visually transparent and homogenous gel is obtained. Let C1 be the resulting gel.

The gel thus obtained is filled into a glass syringe and sterilized by autoclave during 20 min at a temperature of 121° C. Let C2 be the resulting gel.

The viscosity at zero shear rate (=zero viscosity) as well as the elastic modulus G′ and viscous modulus G″ (at 1 Hz frequency) of the hydrogels A1, A2, B1, B2 and C1, C2 are measured by means of a rheometer AR1000 (TA instruments) with a flat geometry of 40 mm, a gap of 1000 microns and an analysis temperature of 25° C.

Hydrogel A1: zero viscosity=189 Pa·s, G′(1 Hz)=203 Pa, G″(1 Hz)=181 Pa

Hydrogel B1: zero viscosity<1 Pa·s, G′(1 Hz)<1 Pa, G″(1 Hz)<1 Pa

Hydrogel C1: zero viscosity=346 Pa·s, G′(1 Hz)=307 Pa, G″(1 Hz)=244 Pa

Hydrogel A2: zero viscosity=52 Pa·s, G′(1 Hz)=87 Pa, G″(1 Hz)=123 Pa

Hydrogel B2: zero viscosity<1 Pa·s, G′(1 Hz)<1 Pa, G″(1 Hz)<1 Pa

Hydrogel C2: zero viscosity=65 Pa·s, G′(1Hz)=112 Pa, G″(1 Hz)=158 Pa

In the hydrogel C1, a synergy between the HA and the alginate can be observed, which makes it possible to have a significantly higher viscosity that that of the hydrogel A1 or of the hydrogel B1 or even of the sum of the viscosities corresponding to hydrogels A1 and B1.

The same synergy is observed with the gels A2, B2 and C2.

The same synergy can be observed in terms of the elastic modulus G′ and of the viscous modulus G″.

### Example 2

### Highlighting of the HA/Alginate Synergy in the Formulation According to the Invention

Preparation of three hydrogels according to the method described below:

### Hydrogel A:

In 30 ml iso-osmolar aqueous solution containing 35 mg/ml sorbitol, 0.30 g sodium hyaluronate at 3.7 MDa is added.

The preparation is mixed by mechanical agitation at room temperature for 24 hours. A visually transparent and homogenous gel is obtained.

The gel thus obtained is filled into a glass syringe and sterilized by autoclave during 5 min at a temperature of 121° C.

### Hydrogel B:

In 30 ml iso-osmolar aqueous solution containing 35 mg/ml sorbitol, 0.03 g sodium alginate at 285 000 Da is added.

The preparation is mixed by mechanical agitation at room temperature for 24 hours. A visually transparent and homogenous gel is obtained.

The gel thus obtained is filled into a glass syringe and sterilized by autoclave during 5 min at a temperature of 121° C.

### Hydrogel C:

In 30 ml iso-osmolar aqueous solution containing 35 mg/ml sorbitol, 0.30 g sodium hyaluronate at 3.7 MDa and 0.03 g sodium alginate at 285 000 Da are added.

The preparation is mixed by mechanical agitation at room temperature for 24 hours. A visually transparent and homogenous gel is obtained.

The gel thus obtained is filled into a glass syringe and sterilized by autoclave during 5 min at a temperature of 121° C.

The viscosity at zero shear rate (=zero viscosity) of the hydrogels A, B and C is measured by means of a rheometer AR1000 (TA instruments) with a flat geometry of 40 mm, a gap of 1000 microns and an analysis temperature of 25° C.

Hydrogel A: zero viscosity=124 Pa·s

Hydrogel B: zero viscosity<1 Pa·s

Hydrogel C: zero viscosity=162 Pa·s

In the hydrogel C, a synergy between the HA and the alginate can be observed, which makes it possible to have a significantly higher viscosity that that of the hydrogel A or of the hydrogel B or even of the sum of the viscosities corresponding to hydrogels A and B.

### Example 3

### Highlighting the HA/Alginate Synergy in the Formulation According to the Invention

Preparation of three hydrogels according to the method described below:

### Hydrogel A:

In 30 ml iso-osmolar aqueous solution containing 1 mg/ml sorbitol, 0.90 g sodium hyaluronate at 440 000 Da is added.

The preparation is mixed by mechanical agitation at room temperature for 24 hours. A visually transparent and homogenous gel is obtained.

### Hydrogel B:

In 30 ml iso-osmolar aqueous solution containing 1 mg/ml sorbitol, 1.2 g sodium alginate at 75 000 Da is added.

The preparation is mixed by mechanical agitation at room temperature for 24 hours. A visually transparent and homogenous gel is obtained.

### Hydrogel C:

In 30 ml iso-osmolar aqueous solution containing 1 mg/ml sorbitol, 0.90 g sodium hyaluronate at 440 000 Da and 1.2 g sodium alginate at 75 000 Da are added.

The preparation is mixed by mechanical agitation at room temperature for 24 hours. A visually transparent and homogenous gel is obtained

The viscosity at zero shear rate (=zero viscosity) of the hydrogels A, B and C is measured by means of a rheometer AR1000 (TA instruments) with a flat geometry of 40 mm, a gap of 1000 microns and an analysis temperature of 25° C.

Hydrogel A: zero viscosity=29 Pa·s

Hydrogel B: zero viscosity<1 Pa·s

Hydrogel C: zero viscosity=42 Pa·s

In the hydrogel C, a synergy between the HA and the alginate can be observed, which makes it possible to have a significantly higher viscosity that that of the hydrogel A or of the hydrogel B or even of the sum of the viscosities corresponding to hydrogels A and B.

### Example 4

### Sterile Aqueous Formulation Injectable in Ophthalmology

In 30 ml of phosphate buffer containing 20 mg/ml sorbitol, 0.90 g sodium hyaluronate at 900 000 Da and 0.6 g sodium alginate at 35 000 Da are added.

The preparation is mixed by mechanical agitation at room temperature for 24 hours. A visually transparent and homogenous gel is obtained.

The gel thus obtained is filled into a glass syringe and sterilized by autoclave during 20 min at a temperature of 121° C. A visually transparent and homogenous gel is obtained.

The pH and osmolarity of the sterilized hydrogel are measured:

pH=6.95 at 25° C.; osmolarity=297 mOsm/kg

The product's zero viscosity is measured by means of a rheometer AR1000 (TA instruments) with a flat geometry of 40 mm, a gap of 1000 microns and an analysis temperature of 25° C.

Zero viscosity=55 Pa·s

The product is easily injectable through a 27 G cannula angled for intraocular use.

A biocompatibility study on pig corneas demonstrates a lack of toxicity towards corneal endothelial cells.

All measured parameters demonstrate that the above formula according to the invention meets the criteria to be used intraocularly.

### Example 5

### Surface Properties of the Formulation According to the Invention/Protective Effect vis-à-vis Eye Tissue

Let A, Band C be the formulations described hereunder:

### Hydrogel A: (Gel According to the Invention)

In 30 ml iso-osmolar aqueous solution, 0.90 g sodium hyaluronate at 1.1 MDa and 0.6 g sodium alginate at 35 000 Da are added.

The preparation is mixed by mechanical agitation at room temperature for 24 hours. A visually transparent and homogenous gel is obtained.

The gel thus obtained is filled into a glass syringe and sterilized by autoclave during 20 min at a temperature of 121° C.

The zero viscosity is measured at 65 Pa·s.

### Hydrogel B: (Gel According to the Invention)

In 30 ml of phosphate buffer, 0.90 g sodium hyaluronate at 3.3 MDa and 0.3 g sodium alginate at 35 000 Da are added.

The preparation is mixed by mechanical agitation at room temperature for 24 hours. A visually transparent and homogenous gel is obtained.

The gel thus obtained is filled into a glass syringe and sterilized by autoclave during 20 min at a temperature of 121° C. A visually transparent and homogenous gel is obtained.

The zero viscosity is measured at 412 Pa·s.

### Hydrogel C: (Gel According to the Prior Art)

In 30 ml iso-osmolar aqueous solution, 0.90 g sodium hyaluronate at 1.1 MDa is added.

The preparation is mixed by mechanical agitation at room temperature for 24 hours. A visually transparent and homogenous gel is obtained.

The gel thus obtained is filled into a glass syringe and sterilized by autoclave during 20 min at a temperature of 121° C.

The zero viscosity is measured at 52 Pa·s.

The steps of cataract surgery are performed on a fresh pig eye, stopping before the step of implantation of an intraocular lens. During this experiment, the surgeon uses either the hydrogel A, B or C (1 ml available for each hydrogel and each experiment).

The experiment is performed 3 times for each hydrogel and the corneal endothelial cells are counted before and after each surgery in order to assess endothelial loss suffered during surgery. For each tested hydrogel, an average of the corneal endothelial loss is performed on the 3 experiments carried out.

A comparison of the endothelial loss is made for the 3 tested hydrogels (normalized at 1 relative to gel C):

Formulation A: 0.79

Formulation B: 0.95

Formulation C: 1

Formulation A according to the invention provides the best protection for the corneal endothelium.

Formulation B according to the invention has a better protection for the corneal endothelium than formulation C (formulation according to the prior art) but this protection is significantly worse than that afforded by formulation A.

### Example 6

### Strong Resistance to Free-Radical Degradation of the Formulation According to the Invention

Let A and B be the formulations described hereunder:

### Hydrogel A: (Gel According to the Invention)

In 30 ml of phosphate buffer, 0.90 g sodium hyaluronate at 900 000 Da and 0.6 g sodium alginate at 35 000 Da are added.

The preparation is mixed by mechanical agitation at room temperature for 24 hours. A visually transparent and homogenous gel is obtained.

The gel thus obtained is filled into a glass syringe and sterilized by autoclave during 20 min at a temperature of 121° C. A visually transparent and homogenous gel is obtained.

### Hydrogel B: (Gel According to the Prior Art)

In 30 ml of phosphate buffer, 0.90 g sodium hyaluronate at 900 000 Da is added.

The preparation is mixed by mechanical agitation at room temperature for 24 hours. A visually transparent and homogenous gel is obtained.

The gel thus obtained is filled into a glass syringe and sterilized by autoclave during 20 min at a temperature of 121° C. A visually transparent and homogenous gel is obtained.

The resistance to free-radical degradation of the gels A and B is compared.

The degradation test is performed using a rheometer AR1000 (TA instruments) with a flat geometry of 40 mm and a gap of 1000 μm.

The degradation test is performed by adding an oxidant (H₂O₂) into the gel to be tested, while homogenizing with a spatula for 1 minute, by placing at a temperature of 37° C. and by applying a deformation of 0.3%. The complex viscosity value η* at 1 Hz is measured at t=5 min and t=40 min.

The formulations tested are compared according to their complex viscosity decrease Δη*(1 Hz) between t=5 min and t=40 min.

| | Δη*(1 Hz) |
|---|---|
| Formulation | between t = 5 min and t = 40 min |
| Gel A | −33% |
| acc. to the invention | |
| Gel B | −37% |
| acc. to the prior art | |

It is observed that the formulation (gel) according to the invention affords a better resistance to free-radical degradation.

Free radicals have a harmful effect vis-à-vis eye tissue. For example, it has been extensively described in the literature that the step of phacoemulsification used during cataract surgery induces a strong release of free radicals. These free radicals create a significant loss of corneal endothelial cells.

Hydrogels based on hyaluronic acid play a key role in protecting the corneal endothelial cells during this surgery since some of the free radicals formed are neutralized.

As demonstrated above, the gel according to the invention has an improved capacity for resisting to degradation due to free radicals. This capacity enables it to have a better preservation of its rheology during cataract surgery, in particular to limit the evolution of viscoelasticity and ocular tissue adhesion properties during the phacoemulsification step. The gel according to the invention thus provides increased protection of the eye tissue and in particular of corneal endothelial cells vis-à-vis free radicals during cataract surgery.

1. Intraocularly injectable sterile aqueous formulation based on a mixture of hyaluronic acid and alginate, or a salt thereof, wherein:
the concentration of hyaluronic acid, or a salt thereof, is comprised between 1% and 4% (weight/volume), and the molecular weight of the hyaluronic acid, or a salt thereof, is comprised between 4×10⁵ Da and 7×10⁶ Da,
the concentration of alginate, or a salt thereof, is comprised between 0.01% and 5% (weight/volume), and
the zero shear viscosity η₀ of said injectable sterile aqueous formulation is comprised between 5 Pa·s and 450 Pa·s

2.-3. (canceled)

4. The aqueous formulation according to claim 1, characterized in that the zero shear viscosity η₀ of said intraocularly injectable sterile aqueous formulation is comprised between 5 and 90 Pa·s.

5. The aqueous formulation according to claim 1, characterized in that the molecular weight of the hyaluronic acid, or a salt thereof, is comprised between 4×10⁵ Da and 2×10⁶.

6. The aqueous formulation according to claim 1, characterized in that the molecular weight of the alginate, or a salt thereof, is less than 3×10⁵ Da.

7. The aqueous formulation according to claim 1, further comprising one or several antioxidants, such as antioxidants from the polyol family.

8. The aqueous formulation according to claim 7, wherein the antioxidant is chosen from the group comprising sorbitol, glycerol, mannitol or propylene glycol.

9. The aqueous formulation according to claim 1, further comprising an anesthetic.

10. The aqueous formulation according to claim 9, wherein the anesthetic is chosen from the group comprising lidocaine alone or in combination with adrenalin, procaine, etidocaine alone or in combination with adrenaline, articaine alone or in combination with adrenaline, mepivacaine, pramocaine, quinisocaine or one or several of these anesthetics.

11. The aqueous formulation according to claim 1, wherein one of the hyaluronic acid salts is sodium hyaluronate, potassium hyaluronate, magnesium hyaluronate, calcium hyaluronate or manganese hyaluronate.

12. The aqueous formulation according to claim 1, wherein one of the alginate salts is sodium alginate.

13. The aqueous formulation according to claim 1, wherein the hyaluronic acid and/or the alginate, or a salt thereof, is reticulated and/or grafted.

14. The aqueous formulation according to claim 1 for its intraocular use in the treatment of ophthalmic diseases.

15. The aqueous formulation according to claim 14, wherein the ophthalmic diseases are from the group comprising retinal degeneration, glaucoma, cataract or a combination of these diseases.

16. Surgical aids and/or temporary implants for use in ophthalmology, wherein they consist, or consist essentially, of an aqueous formulation according to claim 1.

17. Surgical aids and/or temporary implants for use in ophthalmology according to claim 16 for its use during cataract and/or glaucoma surgery.

18. Formulation for tissue protection used in ophthalmology, wherein it consists, or consists essentially, of an aqueous formulation according to claim 1.
